(19) United States
(12) Patent Application Publication
(10) Pub. No.: US 2024/0284091 A1
(43) Pub. Date: Aug. 22, 2024

(54) Ambient Sound Controlled Assembly

(71) Applicant: Ralf Muller, Mississauga (CA)

(72) Inventor: Ralf Muller, Mississauga (CA)

(21) Appl. No.: 18/112,123

(22) Filed: Feb. 21, 2023

(51) Int. Cl. H04R 1/10; A61F 11/14

(52) U.S. Cl. CPC H04R 1/1083; A61F 11/145; H04R 1/1008; H04R 1/1041; H04R 2201/028; H04R 2430/01

(57) ABSTRACT

An ambient sound controlled assembly includes a pair of earmuffs is wearable on a user's head for covering each of the user's ears. Each of the earmuffs is comprised of an acoustically insulating material thereby inhibiting ambient sound from reaching the user's ears when the earmuffs are worn to protect the user's hearing. An audio unit is integrated into the earmuffs and the audio unit has sound detecting capabilities thereby facilitating the audio unit to detect the ambient sound. The audio has sound emitting capabilities for emitting the ambient sound at an adjustable volume. In this way the user can hear the ambient sound in real time at a volume of the user's choosing thereby enhancing comfort for the user in a loud environment.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to noise canceling devices and more particularly pertains to a new noise canceling device for protecting a user's ears from ambient sound. The device includes a pair of earmuffs for protecting a user's ears from ambient sound when the ambient sound is at a dangerously high volume. The device includes a microphone for streaming the ambient sounds and a pair of speakers integrated into the earmuffs for playing the ambient sounds at an adjustable volume.

### (2) Description of Related Art Including Information Disclosed Under 37 CFR 1.97 and 1.98

The prior art relates to noise canceling devices including a variety of noise cancelling headphones that each employs electronic means of countering ambient noise. The prior art discloses a variety of noise cancelling headphones that include a microphone incorporated into an electronic noise cancellation circuit. In no instance does the prior art disclose a pair of earmuffs for hearing protection and an audio unit integrated into the earmuffs for recording ambient sounds and replaying the ambient sounds at a comfortable volume.

### BRIEF SUMMARY OF THE INVENTION

An embodiment of the disclosure meets the needs presented above by generally comprising a pair of earmuffs is wearable on a user's head for covering each of the user's ears. Each of the earmuffs is comprised of an acoustically insulating material thereby inhibiting ambient sound from reaching the user's ears when the earmuffs are worn to protect the user's hearing. An audio unit is integrated into the earmuffs and the audio unit has sound detecting capabilities thereby facilitating the audio unit to detect the ambient sound. The audio has sound emitting capabilities for emitting the ambient sound at an adjustable volume. In this way the user can hear the ambient sound in real time at a volume of the user's choosing thereby enhancing comfort for the user in a loud environment.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of an ambient sound controlled assembly according to an embodiment of the disclosure.

FIG. 2 is a right side view of an embodiment of the disclosure.

FIG. 3 is a front phantom view of an embodiment of the disclosure.

FIG. 4 is a left side view of an embodiment of the disclosure.

FIG. 5 is a perspective view of an embodiment of the disclosure.

FIG. 6 is a perspective view of an alternative embodiment of the disclosure.

FIG. 7 is a right side view of an alternative embodiment of the disclosure.

### DETAILED DESCRIPTION OF THE INVENTION

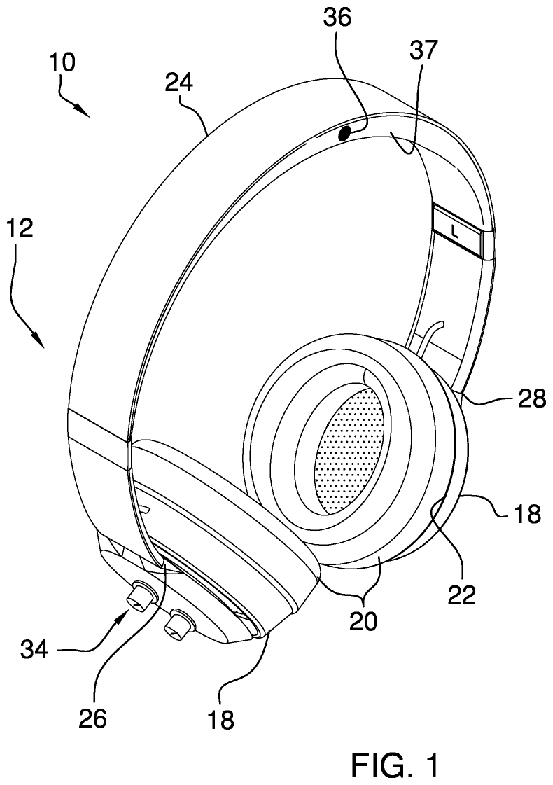

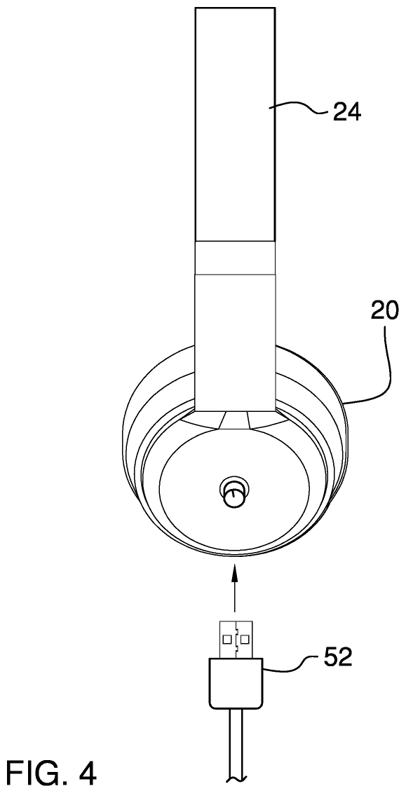

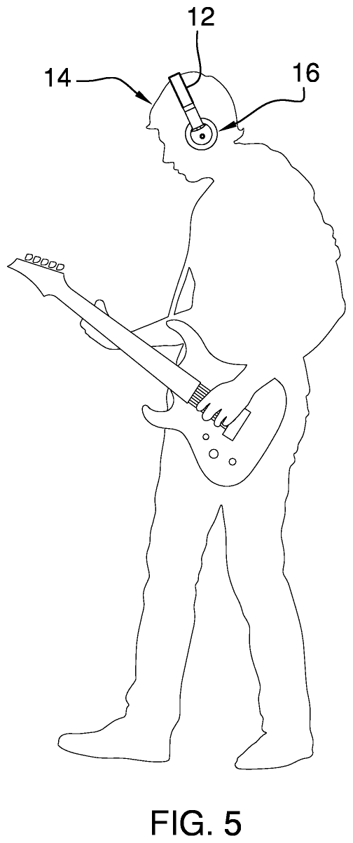

With reference now to the drawings, and in particular to FIGS. 1 through 7 thereof, a new noise canceling device embodying the principles and concepts of an embodiment of the disclosure and generally designated by the reference numeral 10 will be described.

As best illustrated in FIGS. 1 through 7, the ambient sound controlled assembly 10 generally comprises a pair of earmuffs 12 that is wearable on a user's head 14 for covering each of the user's ears 16. Each of the earmuffs 12 is comprised of an acoustically insulating material thereby inhibiting ambient sound from reaching the user's ears 16 when the earmuffs 12 are worn. The acoustically insulating material may be a material that conforms to hearing safety regulations of the Occupational Safety and Health Administration. In this way the earmuffs 12 protect the user's hearing when the user is in the loud environment.

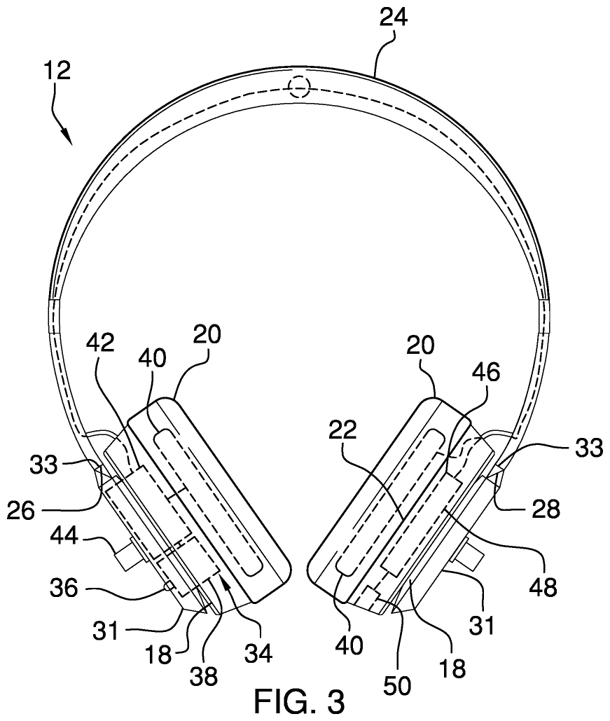

Each of the earmuffs 12 has an outer shell 18 that is concavely arcuate to form a bowl shape and a pad 20 extending around an edge 22 of the bowl shape defined by the outer shell 18 such that the pad 20 abuts the user's head 14 having the outer shell 18 surrounding the user's ear 16. Furthermore, the pair of earmuffs 12 includes a head band 24 that has a first end 26 and a second end 28. Each of the first end 26 and the second end 28 is coupled to the outer shell 18 of a respective one of the earmuffs 12 has the pad 20 associated with each of the earmuffs 12 is directed toward each other. The head band 24 may comprise a central section 30 that slidably engages a pair of outer sections 32 such that the head band 24 has an adjustable length. As is most clearly shown in FIG. 3, the outer shell 18 of each of the earmuffs 12 includes a disk 31 and each of the first end 26 and the second end 28 of the head band 24 is coupled to a perimeter edge 33 of the disk 31 on the outer shell 18 of the respective earmuff 12.

An audio unit 34 is provided and the audio unit 34 is integrated into the earmuffs 12. The audio unit 34 has sound detecting capabilities thereby facilitating the audio unit 34 to stream the ambient sound in real time. The audio unit 34 has sound emitting capabilities and the audio unit 34 emits the ambient sound at an adjustable volume thereby facilitating the user to hear the ambient sound at a volume of the user's choosing. In this way the audio unit 34 enhances comfort for the user in a loud environment.

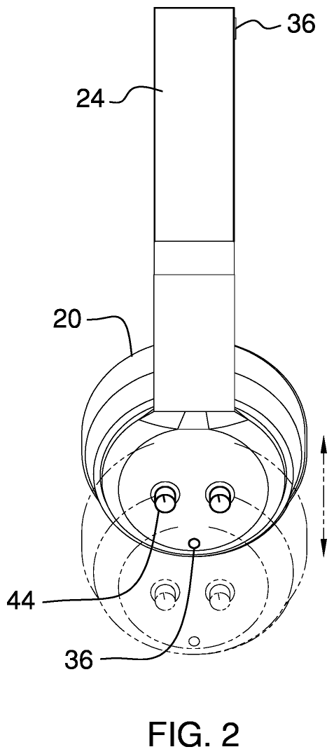

The audio unit 34 comprises a microphone 36 that is integrated into the earmuffs 12 to detect the audible sound. The microphone 36 may comprise an electromagnetic microphone of any conventional design and, as is most clearly shown in FIGS. 1 and 2, the microphone 36 may be integrated into a front edge 37 of the head band 24 at a point that is centrally located between the earmuffs 12. A preamplifier 38 is integrated into the earmuffs 12 and the preamplifier 38 is electrically coupled to the microphone 36 thereby facilitating the preamplifier 38 to amplify an audio signal received from the microphone 36. The preamplifier 38 may comprise a solid state preamplifier of any conventional design that might commonly be employed in microphone circuitry.

The audio unit 34 includes a pair of speakers 40 that is each integrated into a respective one of the earmuffs 12. Each of the speakers 40 is positioned in the bowl shape defined by the outer shell 18 of the respective earmuff 12 to emit audible sound into the users' ears 16 when the user is wearing the earmuffs 12. The audio unit 34 includes an amplifier 42 that is integrated into the earmuffs 12. The amplifier 42 is electrically coupled to the preamplifier 38 thereby facilitating the amplifier 42 to amplify the audio signal received from the preamplifier 38. The amplifier 42 is electrically coupled to each of the speakers 40 thereby facilitating each of the speakers 40 to receive the amplified audio signal from the amplifier 42. Furthermore, the amplifier 42 may comprise a solid state amplifier of any conventional design that might commonly be employed in speaker circuitry.

The audio unit 34 includes a volume knob 44 that is rotatably coupled to the outer shell 18 of a respective one of the earmuffs 12 thereby facilitating the volume knob 44 to be manipulated by the user. The volume knob 44 is electrically coupled to the amplifier 42 and the volume knob 44 adjusts the amplifier 42 between a minimum volume and a maximum volume. In this way the user can adjust the volume of the ambient sound being emitted by the speakers 40.

The audio unit 34 includes a power supply 46 that is integrated into the earmuffs 12 and the power supply 46 is electrically coupled to each of the preamplifier 38 and the amplifier 42. The power supply 46 comprises a rechargeable battery 48 that is positioned in a respective one of the earmuffs 12. The rechargeable battery 48 is electrically coupled to each of the preamplifier 38 and the amplifier 42. The power supply 46 further includes a charge port 50 that is recessed into the outer shell 18 of the earmuff in which the rechargeable battery 48 is positioned. In this way the charge port 50 can insertably receive a charge cord 52 from a charger. The charge port 50 is electrically coupled to the rechargeable battery 48 for charging the rechargeable battery 48.

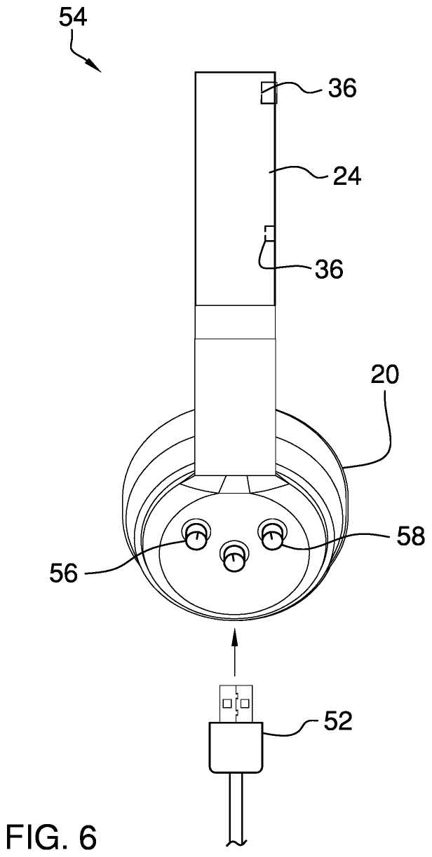

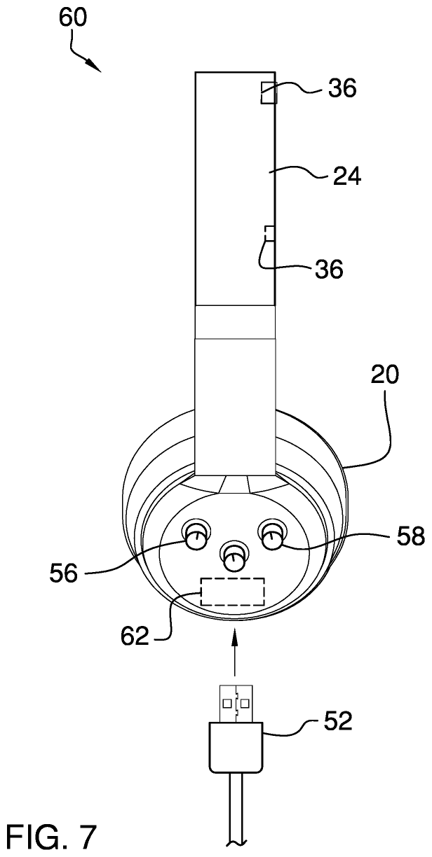

In an alternative embodiment 54 shown in FIG. 6, the audio unit 34 includes a treble knob 56 and a bass knob 58 that are each rotatably coupled to the outer shell 18 of a respective earmuff 12, each electrically coupled to the amplifier 42, for adjusting the treble and bass levels sent to the speakers 40. Continuing in the alternative embodiment 54 in FIG. 6, a pair of the microphones 36 is provided, spaced apart from each other and distributed on the head band 24, to facilitate the audible sound to be detected in stereo. In an alternative embodiment 60 shown in FIG. 7, the audio unit 34 includes an electronic memory 62, in addition to the treble knob 56, the bass knob 58 and the pair of microphones 36, to facilitate the detected audio to be saved in the electronic memory 62.

In use, the earmuffs 12 are worn on the user's head 14 when the user is in a loud environment, such as playing an instrument in a live performance, for example, which involves ambient sound levels that are high enough to cause hearing damage. In this way the earmuffs 12 protect the user's hearing from damage due to high volume sounds. The volume knob 44 is adjusted to facilitate the speakers 40 to emit the ambient sounds in real time at the desired volume. In this way the user can clearly hear the ambient sounds in real time at a level that is comfortable for the user thereby avoiding having their hearing damaged by the ambient sounds.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there only one of the elements.

I claim:

1. An ambient sound controlled assembly for muting and subsequently amplifying ambient noise to a pre-determined sound level, said assembly comprising:
a pair of earmuffs being wearable on a user's head for covering each of the user's ears, each of said earmuffs being comprised of an acoustically insulating material thereby inhibiting ambient sound from reaching the user's ears when said earmuffs are worn wherein said earmuffs are configured to protect the user's hearing; and
an audio unit being integrated into said earmuffs, said audio unit having sound detecting capabilities thereby facilitating said audio unit to detect the ambient sound, said audio having sound emitting capabilities, said audio unit emitting the ambient sound at an adjustable volume thereby facilitating the user to hear the ambient sound in real time at a volume of the user's choosing wherein said audio unit is configured to enhance comfort for the user in a loud environment.

2. The assembly according to claim 1, wherein:
each of said earmuffs has an outer shell being concavely arcuate to form a bowl shape and a pad extending around an edge of said bowl shape defined by said outer wall such that said pad abuts the user's head having said outer shell surrounding the user's ear;
said pair of earmuffs includes a head band having a first end and a second end, each of said first end and said second end being coupled to said outer shell of a respective one of said earmuffs having said pad associated with each of said ear muffs being directed toward each other.

3. The assembly according to claim 1, wherein said audio unit comprising:
a microphone being integrated into said earmuffs wherein said microphone is configured to detect the audible sound; and
a preamplifier being integrated into said earmuffs, said preamplifier being electrically coupled to said microphone thereby facilitating said preamplifier to amplify an audio signal received from said microphone.

4. The assembly according to claim 3, wherein said audio unit includes:
a pair of speakers, each of said speakers being integrated into a respective one of said earmuffs, each of said speakers being positioned in said bowl shape defined by said outer shell of said respective earmuff wherein said pair of speakers is configured to emit audible sound into the users' ears when the user is wearing said earmuffs; and
an amplifier being integrated into said earmuffs, said amplifier being electrically coupled to said preamplifier thereby facilitating said amplifier to amplify the audio signal received from said preamplifier, said amplifier being electrically coupled to each of said speakers thereby facilitating each of said speakers to receive the amplified audio signal from said amplifier.

5. The assembly according to claim 4, wherein said audio unit includes a volume knob being rotatably coupled to said outer shell of a respective one of said earmuffs thereby facilitating said volume knob to be manipulated by the user, said volume knob being electrically coupled to said amplifier, said volume knob adjusting said amplifier between a minimum volume and a maximum volume thereby facilitating the user to adjust the volume of the ambient sound being emitted by said speakers.

6. The assembly according to claim 4, further comprising a power supply being integrated into said earmuffs, said power supply being electrically coupled to each of said preamplifier and said amplifier, said power supply comprising:
a rechargeable battery being positioned in a respective one of said earmuffs, said rechargeable battery being electrically coupled to each of said preamplifier and said amplifier; and
a charge port being recessed into said outer shell of said earmuff in which said rechargeable battery is positioned thereby facilitating said charge port to insertably receive a charge cord from a charger, said charge port being electrically coupled to said rechargeable battery for charging said rechargeable battery.

6. An ambient sound controlled assembly for muting and subsequently amplifying ambient noise to a pre-determined sound level, said assembly comprising:
a pair of earmuffs being wearable on a user's head for covering each of the user's ears, each of said earmuffs being comprised of an acoustically insulating material thereby inhibiting ambient sound from reaching the user's ears when said earmuffs are worn wherein said earmuffs are configured to protect the user's hearing, each of said earmuffs having an outer shell being concavely arcuate to form a bowl shape and a pad extending around an edge of said bowl shape defined by said outer wall such that said pad abuts the user's head having said outer shell surrounding the user's ear, said pair of earmuffs including a head band having a first end and a second end, each of said first end and said second end being coupled to said outer shell of a respective one of said earmuffs having said pad associated with each of said ear muffs being directed toward each other; and
an audio unit being integrated into said earmuffs, said audio unit having sound detecting capabilities thereby facilitating said audio unit to detect the ambient sound, said audio having sound emitting capabilities, said audio unit emitting the ambient sound at an adjustable volume thereby facilitating the user to hear the ambient sound in real time at a volume of the user's choosing wherein said audio unit is configured to enhance comfort for the user in a loud environment, said audio unit comprising:
a microphone being integrated into said earmuffs wherein said microphone is configured to detect the audible sound;
a preamplifier being integrated into said earmuffs, said preamplifier being electrically coupled to said microphone thereby facilitating said preamplifier to amplify an audio signal received from said microphone;
a pair of speakers, each of said speakers being integrated into a respective one of said earmuffs, each of said speakers being positioned in said bowl shape defined by said outer shell of said respective earmuff wherein said pair of speakers is configured to emit audible sound into the users' ears when the user is wearing said earmuffs;
an amplifier being integrated into said earmuffs, said amplifier being electrically coupled to said preamplifier thereby facilitating said amplifier to amplify the audio signal received from said preamplifier, said amplifier being electrically coupled to each of said speakers thereby facilitating each of said speakers to receive the amplified audio signal from said amplifier;
a volume knob being rotatably coupled to said outer shell of a respective one of said earmuffs thereby facilitating said volume knob to be manipulated by the user, said volume knob being electrically coupled to said amplifier, said volume knob adjusting said amplifier between a minimum volume and a maximum volume thereby facilitating the user to adjust the volume of the ambient sound being emitted by said speakers; and
a power supply being integrated into said earmuffs, said power supply being electrically coupled to each of said preamplifier and said amplifier, said power supply comprising:
a rechargeable battery being positioned in a respective one of said earmuffs, said rechargeable battery being electrically coupled to each of said preamplifier and said amplifier; and
a charge port being recessed into said outer shell of said earmuff in which said rechargeable battery is positioned thereby facilitating said charge port to insertably receive a charge cord from a charger, said charge port being electrically coupled to said rechargeable battery for charging said rechargeable battery.

7. A method of adjusting a volume of ambient sounds for a user, the steps of the method comprising:
providing a pair of earmuffs being comprised of an acoustically insulating material thereby facilitating said earmuffs to inhibit ambient sound from reaching a user's ears when said earmuffs are worn;
providing an audio unit being integrated into said earmuffs, said audio unit having a microphone for detecting ambient sounds and speakers for emitting the ambient sounds into the user's ears when said earmuffs are worn;
providing a volume control being movably integrated into said earmuffs, said volume control being electrically coupled to said audio unit for adjusting a volume of said speakers between a minimum volume and a maximum volume;
wearing said earmuffs such that said earmuffs surround each of the user's ears to protect the user's ears from the ambient sound; and
adjusting said volume control to facilitate said speakers to emit the ambient sound at a volume level that is comfortable for the user.
